(19) United States
(12) Patent Application Publication
(10) Pub. No.: US 2024/0168040 A1
(43) Pub. Date: May 23, 2024

(54) BIOLOGICAL SAMPLE PREPARATION AND ANALYSIS

(71) Applicant: DH TECHNOLOGIES DEVELOPMENT PTE. LTD., Singapore (SG)

(72) Inventors: Aaron Stella, Concord (CA); Chang Liu, Concord (CA); Yuguan Liu, Concord (CA); Subhasish Purkayastha, Concord (CA)

(21) Appl. No.: 18/283,375

(22) PCT Filed: Mar. 21, 2022
(86) PCT No.: PCT/IB2022/052566

§ 371 Date: Sep. 21, 2023

Related U.S. Application Data
(60) Provisional application No. 63/164,870, filed on Mar. 23, 2021.

(51) Int. Cl. G01N 33/94; C12Q 1/37; G01N 30/02; G01N 30/72; G01N 33/543

(52) U.S. Cl. CPC G01N 33/94; C12Q 1/37; G01N 30/724; G01N 33/54326; G01N 33/54353; G01N 2030/027; G01N 2333/96477; G01N 2333/976

(57) ABSTRACT

Methods and kits for preparing liquid samples are presently claimed and described. The method may include treating liquid sample with a hydrolysis enzyme, hydrolyzing the liquid sample to prepare a hydrolysate, and purifying the hydrolysate with magnetic based purification. In certain aspects, the hydrolysis enzyme is bound to a magnetic bead or a magnetic particle. Kits for preparing a liquid sample can include a hydrolysis enzyme, magnetic beads or magnetic particles, one or more internal standards, a liquid chromatography column and one or more solvents to be used as mobile phases, one or more calibrant solutions and instructions for use.

### CROSS-REFERENCE TO RELATED APPLICATIONS

This application is related to, and claims the benefit of priority from U.S. Provisional patent application Ser. No. 63/164,870, filed Mar. 23, 2021, and which is incorporated by reference herein in its entirety.

### BACKGROUND

Liquid samples including biological materials and bodily fluids, can often provide critical analytical information. However, many of these liquid samples include a variety of proteins that may hinder downstream analysis of small molecules. Additionally target analytes may undergo metabolism including glucuronidation, resulting in complexes that may prove challeding the analyze directly. To achieve a more accurate analysis of liquid samples, proper sample preparation is critical. Typical sample preparation methods may require significant time and specific conditions and are not easily automated.

### SUMMARY

The inventors have recognized the need for a sample preparation method that efficiently extracts protein from liquid samples before downstream analysis. In particular, a sample preparation method that employs both hydrolysis and magnetic bead purification.

One aspect of the disclosure relates to a method of preparing a liquid sample, comprising the steps of treating the liquid sample with a hydrolysis enzyme, hydrolyzing the liquid sample to prepare a hydrolysate, and purifying the hydrolysate with magnetic based purification. In one aspect, the liquid sample is a biological sample. In another aspect, the biological sample is selected from the group consisting of urine, blood, oral fluid, and plasma. In yet another aspect, the hydrolysis enzyme is bound to a magnetic bead or a magnetic particle.

In one aspect, the magnetic based purification utilizes magnetic beads or magnetic particles. In a further aspect, the magnetic based purification further comprises the steps of precipitating proteins and any excess hydrolysis enzyme in the hydrolysate with a precipitating reagent, incubating the magnetic beads or magnetic particles with the precipitated proteins for a time sufficient to produce a suspension, and magnetically separating the magnetic beads or magnetic particles from the suspension to produce a supernatant. In one aspect, the precipitating reagent is selected from the group consisting of zinc salts, zinc sulfate, glycols, alcohols, acids, sulfates, acids, acetonitrile, and combinations thereof. In a further aspect, the precipitating reagent is 0.4M zinc sulfate.

In some aspects, the magnetic based purification is based on immunopurification or affinity purification. In another aspect, the magnetic bead or magnetic particle comprises a monoclonal antibody, a polyclonal antibody, a synthetic antibody mimic, an aptamer, an affimer, DARPins, or oligonucleotides or peptides that bind to specific targets with high affinity. In some aspects, the magnetic bead or magnetic particle comprises streptavidin, and the hydrolysis enzyme comprises biotin. In some aspects, the magnetic based purification is based on ion-exchange. In yet another aspect, the magnetic beads or magnetic particles have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate.

In some aspects, the liquid sample further comprises an internal standard. In another aspect, the hydrolysis enzyme is capable of hydrolyzing glycosidic linkages. In yet another aspect, the hydrolysis enzyme is β-glucuronidase, trypsin, chymotrypsin, a protease, LysC, LysN, AspN, GluC, ArgC, pronase, pepsin, prolidase. In a further aspect, the hydrolysis enzyme is capable of hydrolyzing one or more linkages in a metabolite such as, for example, an opioid metabolite (e.g., codeine-6-glucuronide and morphine-6-glucuronide linkages).

In some aspects, the hydrolyzing step is performed for up to about 20 minutes. In some aspects, this step is performed for about 10 minutes to about 20 minutes. In a further aspect, the hydrolyzing step is performed for about 15 minutes. In another aspect, the hydrolyzing step is performed at a temperature of up to 60° C. In some aspects, this step is performed at about 50° C. to about 60° C. In yet another aspect, the aqueous biological sample is hydrolyzed at a temperature of about 55° C.

In some aspects, the incubating further comprises mixing, shaking, or vortexing the magnetic beads or magnetic particles with the hydrolysate.

In some aspects, the method further comprises aliquoting the supernatant. In yet another aspect, the aliquoted supernatant is separated and/or enriched using a chromatography instrument, microflow, or solid phase extraction. In some aspects, the chromatography instrument is a high performance liquid chromatography (HPLC) instrument or an ultra high performance liquid chromatography instrument (UPLC). In a further aspect, the aliquoted supernatant is separated and/or enriched using a trap-and-elute workflow.

In some aspects, the aliquoted supernatant is acoustically injected into an open port interface and transferred to an ionization source or directly injected into an ionization source. In another aspect, the ionized supernatant is analyzed with a mass spectrometer. In some aspects, ions of interest are selected from the ionized supernatant using differential mobility spectrometry prior to analyzing the ionized supernatant with a mass spectrometer.

In some aspects, the method improves the efficiency of the liquid sample hydrolysis or improves the liquid sample hydrolysate quality. In another aspect, the improved quality of the hydrolysate includes a reduction in carryover. In yet another aspect, the method is used in an automated and/or continuous process of preparing a liquid sample.

In some aspects, the method is used to prepare a liquid sample for clinical analysis. In some aspects, the clinical analysis is used to screen for drugs of abuse. In a further aspect, the screened drugs of abuse are selected from the group consisting of amphetamines, methamphetamines, benzodiazepines, barbiturates, marijuana, cocaine, PCP, methadone, and opioids (narcotics).

One aspect of the disclosure relates to a kit for preparing a liquid sample, wherein the kit comprises:
a hydrolysis enzyme selected from the group consisting of β-glucuronidase, trypsin, chymotrypsin, proteases, LysC, LysN, AspN, GluC, ArgC, pronase, pepsin, prolidase, and a biotinylated enzyme, and
magnetic beads or magnetic particles, wherein the magnetic beads or magnetic particles comprise a monoclonal antibody, a polyclonal antibody, streptavidin, or have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate. In some aspects, the kit may include one or more internal standards, and/or a liquid chromatography column and/or one or more solvents to be used as mobile phases, one or more calibrant solutions, and instructions for use.

Other aspects and features of the present disclosure will become apparent to those ordinarily skilled in the art upon review of the following description of specific embodiments of the disclosure in conjunction with the accompanying figures.

### BRIEF DESCRIPTION OF THE FIGURES

Embodiments of the present disclosure will now be described, by way of example only, with reference to the attached Figures, wherein:

FIGS. 1A-1E illustrate isobaric chromatograms for drugs of abuse according to one embodiment of the disclosure.

FIG. 1A illustrates an embodiment of the disclosure where peak identities are morphine, hydromorphone, norcodeine, and norhydrocodone.

FIG. 1B illustrates an embodiment of the disclosure where peak identities are codeine and hydrocodone.

FIG. 1C illustrates an embodiment of the disclosure where peak identities are oxymorphone, dihydrocodeine, and noroxycodone.

FIG. 1D illustrates an embodiment of the disclosure where peak identities are 6-Monoacetylmorphine (6-MAM), a unique metabolite of heroin, and naloxone.

FIG. 1E illustrates an embodiment of the disclosure where peak identities are norclozapine and flunitrazepam.

FIG. 2A illustrates an enzyme conjugated to a magnetic bead.

FIG. 2B illustrates the chemical immobilization of f3-Glucuronidase to a magnetic bead.

FIG. 3 is a graphical representation of the pressure tracings from liquid samples hydrolyzed according to an embodiment.

### DETAILED DESCRIPTION

It is to be understood that this disclosure is not limited to the particular methodology, protocols, and reagents described herein and as such may vary. It is also to be understood that the terminology used herein is for the purpose of describing particular embodiments only and is not intended to limit the scope of the present disclosure or the appended claims.

As used herein and in the appended claims, the singular forms “a,” “an,” and “the” include plural reference unless the context clearly indicates otherwise.

The term “about” is used in connection with a numerical value throughout the specification and the claims denote an interval of accuracy, familiar and acceptable to a person skilled in the art. In general, such an interval of accuracy is +/−10%.

Unless defined otherwise, all technical and scientific terms used herein have the same meaning as commonly understood to one of ordinary skill in the art to which this disclosure belongs.

Embodiments of the disclosure include methods of preparing a liquid sample. The liquid sample may be a biological sample. Biological samples may be biological fluids, which may include, but are not limited to, blood, plasma, serum, oral fluid, or other bodily fluids or excretions, such as but not limited to saliva, urine, cerebrospinal fluid, lacrimal fluid, perspiration, gastrointestinal fluid, amniotic fluid, mucosal fluid, pleural fluid, sebaceous oil, exhaled breath, and the like.

In an embodiment, the method of preparing a liquid sample includes hydrolyzing the liquid samples. In some embodiments, an internal standard is added before hydrolysis, but it can be appreciated by one of ordinary skill that the internal standard may be added after hydrolysis is completed.

Hydrolysis can be chemical or enzymatic. Enzymatic hydrolysis is a process where peptide bonds in proteins are hydrolyzed using enzymes, such as proteases, peptidases, or peptide hydrolases. Proteases can be either exopeptidases, which act near the end of a polypeptide chain and include, for example, aminopeptidases and dipeptidyl peptidases, or endopeptidases, which act on nonterminal peptide bonds and include, for example, serine proteases, cysteine proteases, aspartic acid proteases, and metallo endopeptidases.

In some embodiments, the method includes treating the liquid sample with a hydrolysis enzyme to prepare a hydrolysate. Suitable hydrolysis enzymes include, but are not limited to, β-glucuronidase, trypsin, chymotrypsin, a protease, LysC, LysN, AspN, GluC, ArgC, pronase, pepsin, and prolidase. Suitable hydrolysis enzymes also include those that are capable of hydrolyzing glycosidic linkages, such as those formed during metabolic processes. Non-limiting examples of these glycosidic linkages include codeine-6-glucuronide and morphine-6-glucuronide linkages.

The hydrolysis enzyme may be incubated with the liquid sample for a suitable amount of time. Suitable amounts of time may be dependent on the hydrolysis enzyme used and the liquid sample being hydrolyzed. Non-limiting examples of hydrolysis incubation times include about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some methods, hydrolysis incubation times of greater than about 20 minutes may be employed.

The hydrolysis enzyme may be incubated with the liquid sample at a suitable temperature. Suitable temperatures may be dependent on the hydrolysis enzyme used and the liquid sample being hydrolyzed. Non-limiting examples of hydrolysis temperatures include about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In an embodiment, the hydrolysis enzyme is incubated with the biological sample for about 20 minutes at about 55° C.

In an embodiment, the method of preparing a liquid sample includes purifying the hydrolysate with magnetic based purification. In some embodiments, the magnetic based purification utilizes magnetic bead or magnetic particles. Magnetic beads or magnetic particles are typically nanoparticles or microparticles that have paramagnetic properties. Magnetic beads or magnetic particles are typically hydrophilic and disperse easily in aqueous solutions. The surface coating and/or chemistry of the magnetic beads or magnetic particles allow various biomolecules such as proteins, peptides, and nucleic acids to bind to the magnetic beads or magnetic particles. Once a biomolecule of interest is bound to a magnetic bead or magnetic particle, magnetic separation is employed to the magnetic beads or magnetic particles from a suspension by applying a magnetic force.

In an embodiment, the hydrolysis enzyme is bound to magnetic beads or magnetic particles. In an embodiment, the hydrolysis enzyme is chemically bonded to the magnetic bead or magnetic particle. In an exemplary embodiment, subjecting the liquid sample to hydrolysis in the presence of the immobilized hydrolysis enzyme generates a hydrolyzed liquid sample that can be acoustically ejected in an open port interface and transferred to an ionization source or directly injected into an ionization source.

In an embodiment, the magnetic based purification is based on immunopurification or affinity purification. In these embodiments, the magnetic beads or magnetic particles may comprise a monoclonal antibody, a polyclonal antibody, a synthetic antibody mimic, an aptamer, an affimer, DARPins, or oligonucleotides or peptides that bind to specific targets with high affinity. In an embodiment, the magnetic based purification employs the streptavidin-biotin system, where the magnetic bead or magnetic particle comprises streptavidin, and the hydrolysis enzyme comprises biotin. In an exemplary embodiment, the liquid sample is hydrolyzed using the biotinylated enzyme. After hydrolysis is complete, the biotinylated enzyme is affinity captured using a streptavidin-coated magnetic bead or magnetic particle. The resulting hydrolyzed liquid sample can be acoustically ejected in an open port interface and transferred to an ionization source or directly injected into an ionization source.

In an embodiment, the magnetic bead purification is based on ion-exchange. In these embodiments, the magnetic beads or magnetic particles have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate.

In an embodiment, the magnetic bead purification step comprises precipitating the proteins in the hydrolysate and/or and any excess hydrolysis enzyme. Non-limiting examples of precipitation methods include changes in temperature, pH, and/or salt concentrations or the addition of a precipitating reagent. See, e.g., Metz, Clyde, Chemistry: Inorganic Qualitative Analysis in the Laboratory, 1980. In an embodiment, the proteins in the hydrolysate are precipitated using a precipitating reagent. Non-limiting examples of precipitating reagents include zinc salts, zinc sulfate, glycols, alcohols, acids, sulfates, acids, acetonitrile. Precipitating reagents may be used alone or in combination with other precipitating reagents or other organic solvents. In one embodiment, the precipitating reagent is 0.4M zinc sulfate.

In an embodiment, the magnetic bead purification step also comprises incubating the magnetic beads or magnetic particles with the precipitated proteins for a time sufficient to produce a suspension. Suitable amounts of time may be dependent on the type of magnetic bead purification being employed. In some embodiments, the time sufficient is less than 10 minutes, alternatively less than 9 minutes, alternatively less than 8 minutes, alternatively less than 7 minutes, alternatively less than 6 minutes, alternatively less than 5 minutes, alternatively less than 4 minutes, alternatively less than 3 minutes, alternatively less than 2 minutes, alternatively less than 1 minute.

In an embodiment, the magnetic bead purification step also comprises magnetically separating the magnetic beads or magnetic particles from the suspension to produce a supernatant. Magnetic separation may include applying a magnetic field to the magnetic beads or magnetic particles, which will draw the magnetic beads or magnetic particles toward the side wall of a sample container. The magnetic field may be applied using a magnetic rack. This allows for the removal of a biomolecule of interest bound to the magnetic beads or magnetic particles from the suspension and results in a supernatant that can be aliquoted and analyzed.

In an embodiment of the disclosure, an analyzer may be used to analyze aliquoted supernatant. The term “analyzer” may include any suitable instrument capable of analyzing a sample such as a biological sample. Examples of analyzers include chromatography instruments, mass spectrometers, immunoanalyzers, hematology analyzers, microbiology analyzers, and/or molecular biology analyzers.

In an embodiment, the aliquoted supernatant is separated and/or enriched using a chromatography instrument, microflow, or solid phase extraction. Examples of chromatography instruments, include, but is not limited to, a liquid chromatography instrument such as a high-performance liquid chromatography (HPLC) instrument or an ultra-high performance chromatography (UHPLC) instrument.

In an embodiment, the aliquoted supernatant is analyzed using a mass spectrometer. The aliquoted supernatant may be introduced into the mass spectrometer with additional separation or without additional separation. If separation is not required, the aliquoted supernatant may be acoustically injected into an open port interface and transferred to an ionization source or directly injected into an ionization source. Separation and/or enrichment done prior to mass spectrometry analysis may include, but is not limited to, liquid chromatography, microflow, and solid phase extraction. In some embodiment, a trap-and-elute workflow may be used.

In an embodiment, ions of interest are selected from the ionized supernatant using differential mobility spectrometry prior to analyzing the ionized supernatant with a mass spectrometer. Differential mobility spectrometry enables the separation of coeluting compounds, isobaric compounds, isomeric compounds, constitutional isomers, or diastereomers.

In an embodiment, the method is used to prepare a liquid sample for clinical analysis. The clinical analysis can be used to screen for drugs of abuse. Non-limiting examples of drugs of abuse include amphetamines, methamphetamines, benzodiazepines, barbiturates, marijuana, cocaine, PCP, methadone, and opioids (narcotics). In an embodiment, the clinical analysis is a clinical urine test or a urinalysis, and is the analysis is used to screen for drugs of abuse. Urine is a common biological sample used in testing for drugs of abuse. A urinalysis or clinical urine test can detect the presence of a drug of abuse after the drug effects have worn off.

One embodiment of the disclosure includes a kit for preparing a liquid sample for analysis. The kit comprises a hydrolysis enzyme. Non-limiting examples of the hydrolysis enzyme include β-glucuronidase, trypsin, chymotrypsin, a protease, LysC, LysN, AspN, GluC, ArgC, pronase, pepsin, prolidase, or a biotinylated enzyme. The kit also includes magnetic beads or magnetic particles. In some embodiments, the magnetic bead or magnetic particles may comprise a monoclonal antibody, a polyclonal antibody, streptavidin, or have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate. The kit may further include one or more internal standards. In some embodiments, the internal standard may be added in a constant amount to samples, the blank, and calibration standards. The internal standard may be a compound that is highly similar to an analyte of interest. The kit may also include a liquid chromatography column. The liquid chromatography column may be a reversed-phase chromatography column. Non-limiting examples of reversed-phase chromatography columns include C18-bonded silica, C8-bonded silica, pure silica, cyano-bonded silica, and phenyl-bonded silica. The kit may also include one or more solvents. Non-limiting examples of suitable chromatography solvents include organic solvents, such as acetonitrile, methanol, and propanol. To improve the chromatographic peak shape, acids, such as formic acid, triflouroacetic acid, or acetic acid, may be included. These acids also provide a source of protons in reverse phase LC/MS applications. The kit may also include one or more calibrant or calibration solution. The calibrant or calibration solution may be used to calibrate an analyzer. The kit may also include instructions for use.

### Examples

### Example 1: Exemplary Sample Preparation and Analysis Method

Urine samples can be collected according to the SAMHSA guidelines (Substance Abuse and Mental Health Services Administration Center for Substance Abuse Prevention), available at https://www.samhsa.gov/sites/default/files/specimen-collection-handbook-2014.pdf. Urine specimens are typically submitted to certified laboratories within 24 hours after collection.

A 30 μL aliquot of an internal standard, 80 μL of water, 40 μL of a hydrolysis enzyme, and 20 μL of urine sample are added to a plastic reaction vessel. The urine sample is mixed via vortexing and is subsequently hydrolyzed by a fast-acting enzyme that is proven capable of hydrolyzing glycosidic linkages, including codeine-6-glucuronide and morphine-6-glucuronide, in 15 minutes at 55° C. Magnetic beads (1 mg/mL) in the amount of 25 μL, and 105 μL of water, and 30 μL of 0.4M zinc sulfate are then added to the reaction milieu. After vortexing briefly, a magnetic rack is then used to pull the magnetic beads with the precipitated proteins to one side of the reaction vessel, leaving behind a very clear supernatant that contains the analyte(s) of interest. This supernatant can then be easily aliquoted into an HPLC vial or directly injected into a mass spectrometer.

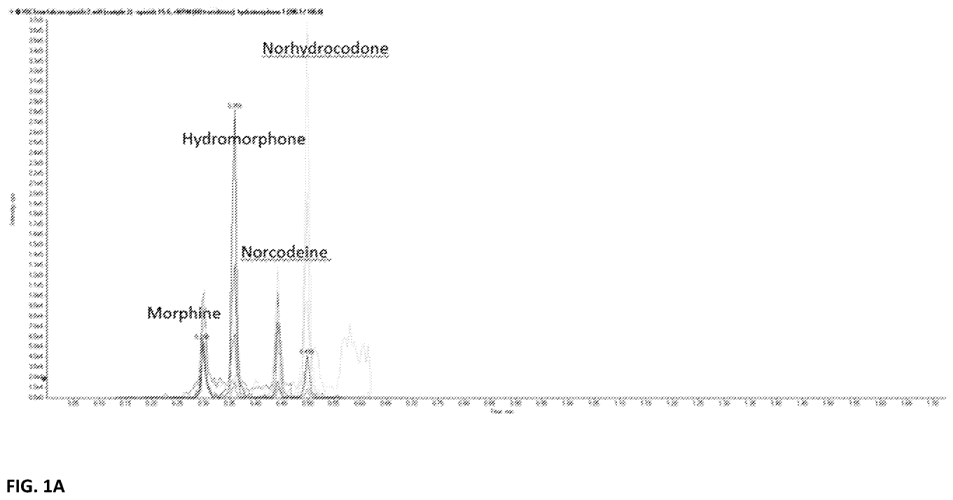

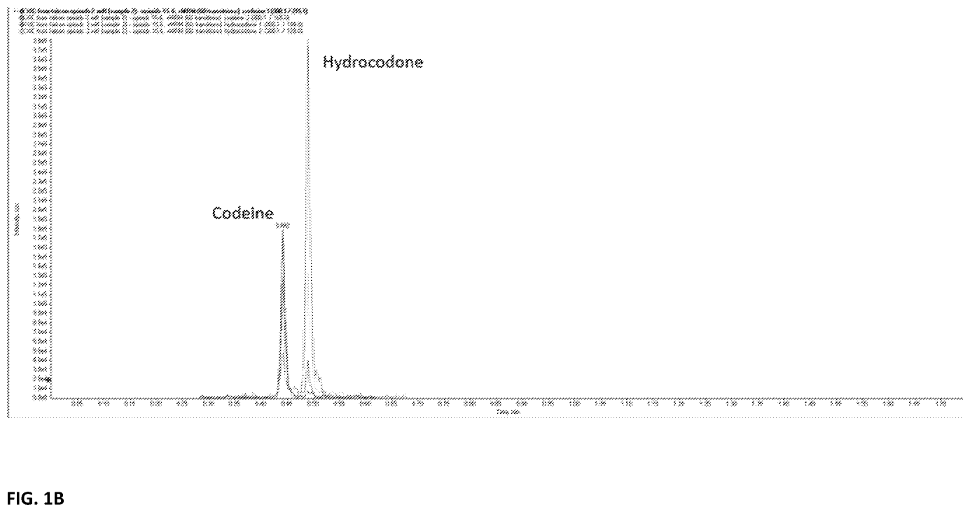

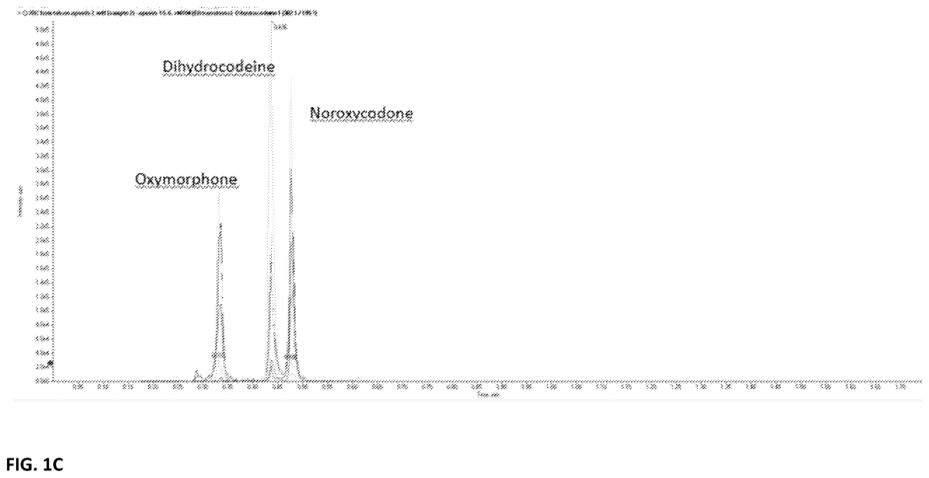

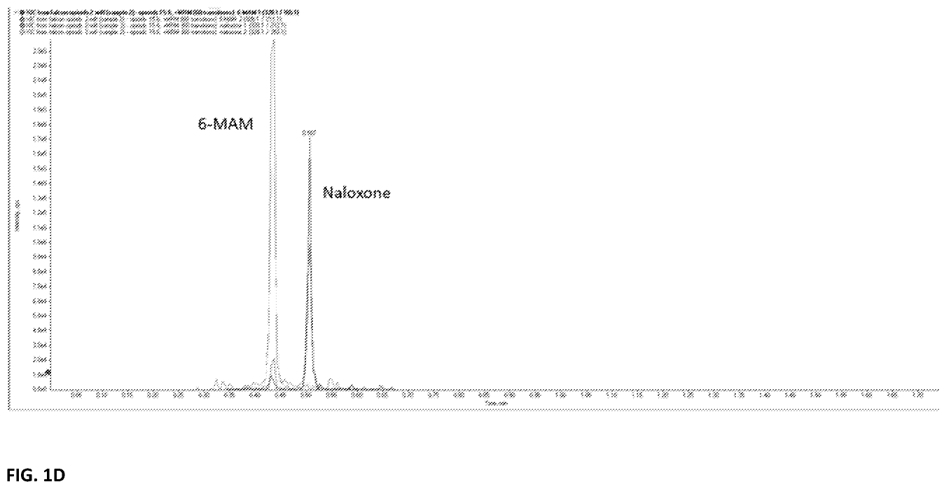

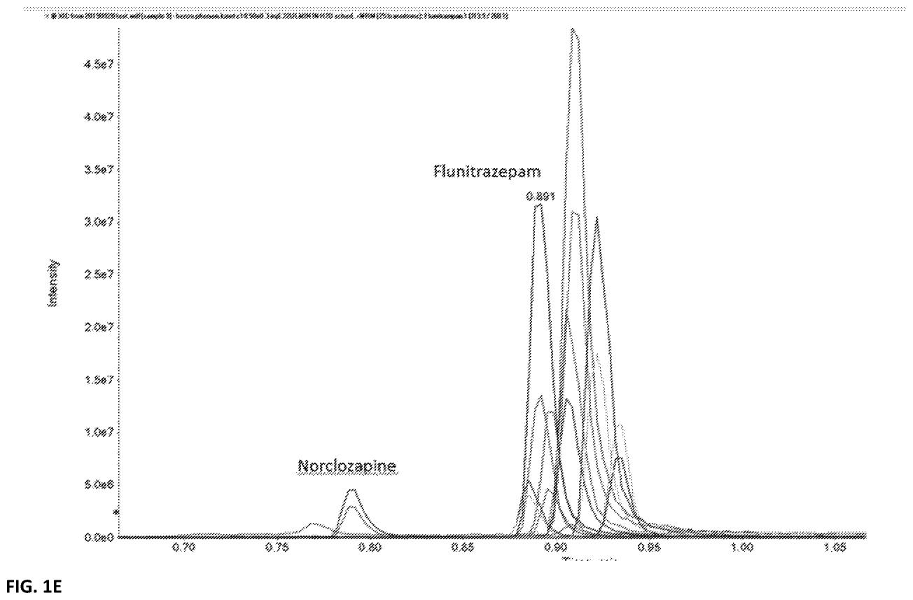

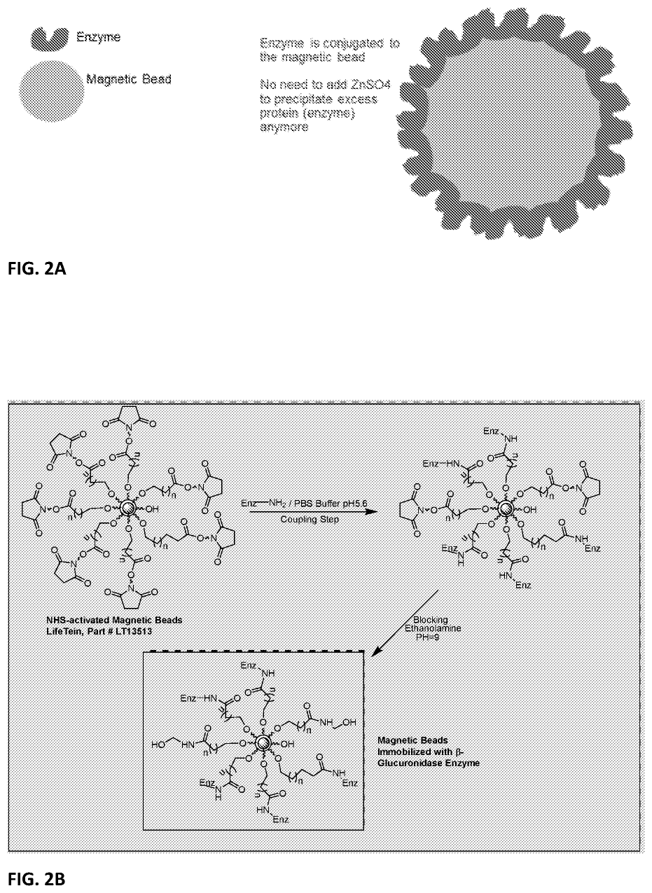

Chromatograms of selected samples are illustrated in FIGS. 1A-1E and identify the presence of morphine, hydromorphone, norcodeine, norhydrocodone (FIG. 1A), codeine and hydrocodone (FIG. 1B), oxymorphone, dihydrocodeine, and noroxycodone (FIG. 1C), 6-Monoacetylmorphine (6-MAM) and naloxone (FIG. 1D) and norclozapine and flunitrazepam (FIG. 1E).

### Example 2: On-Bead Hydrolysis of Liquid Samples

Calibration curves and quality control samples for morphine and codeine were run using both a traditional hydrolysis method and an on-bead hydrolysis method. The study samples were urine samples were spiked with morphine-6-glucuronide and codeine-6-glucuronide. Table 1 illustrates the target concentrations.

TABLE 1

| Hyd Control | Gluc, ng/ml | Free, ng/ml |
|---|---|---|
| Codeine-6-Gluc | 15000 | 9444 |
| Morphine-3-Gluc | 15000 | 9275 |

The test samples were run using the traditional method, with and without adding a hydrolysis enzyme. The test samples were also run using the hydrolysis enzyme-on-bead method. The results from incubating these samples at 55° C. for 15 and 60 minutes are shown in Table 2.

TABLE 2
55° C., 15 and 60 min., 900 rpm agitation

| On Bead Method | Morphine | Accuracy (%) | Codeine | Accuracy (%) |
|---|---|---|---|---|
| On Bead Hydrolysis 1-15 min | 13155.63 | 141.84 | 3789.09 | 40.12 |
| On Bead Hydrolysis 2-15 min | 11784.82 | 127.06 | 3502.00 | 37.08 |
| On Bead Hydrolysis 1-60 min | 9966.9 | 107.46 | 6101.15 | 64.6 |
| On Bead Hydrolysis 2-60 min | 10412.75 | 112.27 | 6153.89 | 65.16 |

The glucuronide was cleaved from the morphine-6-glucuronide molecule efficiently enough to produce concentrations within the acceptable range. The glucuronide was also cleaved from the codeine-6-glucuronide molecule, but with less efficiency in a time-dependent manner. A clean sample, suitable for reliable acoustic or direct injections was also produced with the magnetic beads. There is no need to precipitate excess enzyme as the enzyme is conjugated to the beads and would not be injected into a liquid chromatography instrument or ionization source. As a result, a precipitation step during sample preparation was eliminated (saving time and reagent), and a highly-stable and reproducible column pressure profile was achieved throughout the study.

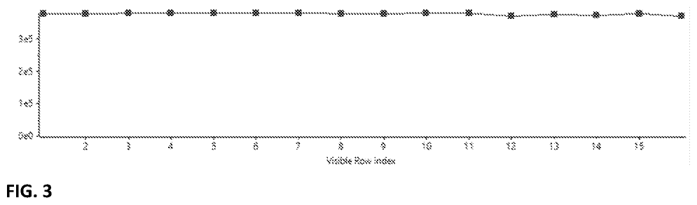

FIG. 3 is a graphical representation of the pressure tracings of each individual sample that was analyzed during the study. Table 3 summarizes the average pressure tracing's area value, its standard deviation, and its very low coefficient of variation value over the 16-sample study.

TABLE 3

| Mean | 3.78E+05 |
|---|---|
| SD | 3251.03 |
| % CV | 0.86 |

While the present disclosure has been described with reference to certain embodiments, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular embodiments disclosed, but that the present disclosure will include all aspects falling within the scope of the appended claims.

All patents, patent applications, publications, and descriptions mentioned above are herein incorporated by reference in their entirety.

1. A method of preparing a liquid sample, comprising:
treating the liquid sample with a hydrolysis enzyme,
hydrolyzing the liquid sample to prepare a hydrolysate, and
purifying the hydrolysate with magnetic based purification.

2. The method of claim 1, wherein the liquid sample is a biological sample.

3. The method of claim 2, wherein the biological sample is selected from the group consisting of urine, blood, oral fluid, and plasma.

4. The method claim 1, wherein
the hydrolysis enzyme is bound to a magnetic bead or a magnetic particle.

5. The method of claim 1, wherein
the magnetic based purification utilizes magnetic beads or magnetic particles.

6. The method of claim 5, wherein the magnetic based purification further comprises the steps of:
precipitating proteins and/or any excess hydrolysis enzyme in the hydrolysate with a precipitating reagent,
incubating the magnetic beads or magnetic particles with the precipitated proteins for a time sufficient to produce a suspension, and
magnetically separating the magnetic beads or magnetic particles from the suspension to produce a supernatant.

7. The method of claim 6, wherein the precipitating reagent is selected from the group consisting of zinc salts, zinc sulfate, glycols, alcohols, acids, sulfates, acids, acetonitrile, and combinations thereof.

8. The method of claim 7, wherein the precipitating reagent is 0.4M zinc sulfate.

9. The method of claim 4, wherein the magnetic based purification is based on immunopurification or affinity purification.

10. The method of claim 9, wherein the magnetic bead or magnetic particle comprises a monoclonal antibody, a polyclonal antibody, a synthetic antibody mimic, an aptamer, an affimer, DARPins, or oligonucleotides or peptides that bind to specific targets with high affinity.

11. The method of claim 10, wherein the magnetic bead or magnetic particle comprises streptavidin and the hydrolysis enzyme comprises biotin.

12. The method of claim 4, wherein the magnetic based purification is based on ion-exchange.

13. The method of claim 12, wherein the magnetic beads or magnetic particles have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate.

14. The method of claim 1, wherein the liquid sample further comprises an internal standard.

15. The method of claim 1, wherein the hydrolysis enzyme is capable of hydrolyzing glycosidic linkages.

16. The method of claim 1, wherein the hydrolysis enzyme is β-glucuronidase, trypsin, chymotrypsin, a protease, LysC, LysN, AspN, GluC, ArgC, pronase, pepsin, prolidase.

17. The method of claim 1, wherein the hydrolysis enzyme is capable of hydrolyzing codeine-6-glucuronide and morphine-6-glucuronide linkages.

18. The method of claim 1, wherein the hydrolyzing step is performed for up to 20 minutes.

19. The method of claim 18, wherein the hydrolyzing step is performed for about 15 minutes.

20. The method of claim 1, wherein the hydrolyzing step is performed at a temperature of up to about 60° C.

21. The method of claim 20, wherein the aqueous biological sample is hydrolyzed at a temperature of about 55° C.

22. The method of claim 3, wherein the incubating further comprises mixing, shaking, or vortexing the magnetic beads or magnetic particles with the hydrolysate.

23. The method of claim 6, further comprising aliquoting the supernatant.

24. The method of claim 23, wherein the aliquoted supernatant is separated and/or enriched using a chromatography instrument, microflow, solid phase extraction.

25. The method of claim 24, wherein the chromatography instrument is a high performance liquid chromatography (HPLC) instrument or an ultra high performance liquid chromatography instrument (UPLC).

26. The method of claim 24, wherein the aliquoted supernatant is separated and/or enriched using a trap-and-elute workflow.

27. The method of claim 23, wherein the aliquoted supernatant is acoustically injected into an open port interface and transferred to an ionization source or directly injected into an ionization source.

28. The method of claim 27, wherein the ionized supernatant is analyzed with an analyzer.

29. The method of claim 28, wherein the analyzer is a mass spectrometer.

30. The method of claim 29, wherein ions of interest are selected from the ionized supernatant using differential mobility spectrometry prior to analyzing the ionized supernatant with a mass spectrometer.

31. The method of claim 1, wherein the method is used to prepare a liquid sample for clinical analysis.

32. The method of claim 31, wherein the clinical analysis is used to screen for drugs of abuse.

33. The method of claim 32, wherein the drugs of abuse are selected from the group consisting of amphetamines, methamphetamines, benzodiazepines, barbiturates, marijuana, cocaine, PCP, methadone, and opioids (narcotics).

34. A kit for preparing a liquid sample, wherein the kit comprises:
a hydrolysis enzyme selected from the group consisting of β-glucuronidase, trypsin, chymotrypsin, proteases, LysC, LysN, AspN, GluC, ArgC, pronase, pepsin, prolidase, and a biotinylated enzyme, and
magnetic beads or magnetic particles, wherein the magnetic beads or magnetic particles comprise a monoclonal antibody, a polyclonal antibody, streptavidin, or have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate,
one or more internal standards, a liquid chromatography column, and one or more solvents to be used as mobile phases, one or more calibrant solutions, and instructions for use.
